(19) United States
(12) Patent Application Publication
(10) Pub. No.: US 2010/0158193 A1
(43) Pub. Date: Jun. 24, 2010

(54) Interventional Devices Formed Using Compositions Including Metal-Coated Nanotubes Dispersed In Polymers, And Methods Of Making And Using Same

(76) Inventor: Mark C. Bates, Carlsbad, CA (US)

Correspondence Address: JONES DAY, 222 EAST 41ST ST, NEW YORK, NY, 10017, US

(21) Appl. No.: 12/342,013

(22) Filed: Dec. 22, 2008

(51) Int. Cl. G01N 23/04; A61M 25/10; B29C 47/00; B05D 3/00

(52) U.S. Cl. 378/62; 604/103.08; 264/209.1; 427/2.1

(57) ABSTRACT

A catheter, formed in part of a polymer with a plurality of metal-coated nanotubes dispersed therein, is provided, along with methods of making and using such catheters. The method includes heating a polymer above a melting point of the polymer; dispersing a plurality of metal-coated nanotubes within the polymer; and forming the polymer having the plurality of metal-coated nanotubes dispersed therein into a catheter component. Methods of imaging a catheter including a polymer having a plurality of metal-coated nanotubes dispersed therein also is provided, which includes inserting a portion of a catheter into a body lumen; exposing the portion of the catheter to x-ray radiation; and obtaining an x-ray image of the portion of the catheter.

### FIELD

The present invention generally relates to interventional devices such as dilatation catheters having enhanced visibility when viewed with radiographic imaging systems, and methods for making and using such devices.

### BACKGROUND

Interventional techniques have been developed wherein catheters are used to perform diagnostic and therapeutic procedures, such as stenting and angioplasty. As the medical field grows increasingly advanced, there is a growing need for precision instruments and devices. In particular, the extension of diagnostic and treatment modalities has been limited by the inability to access smaller vessels, such as those presented by the cerebrovasculature and distal coronary systems. Similarly, lower profile angioplasty and stent delivery devices are becoming increasingly important as the number of patients presenting with complex calcium dominant coronary lesions continued to increase in the aging population.

In particular, contrast agents typically used to inflate balloon catheters are relatively viscous. At smaller catheter sizes, difficulties arise due to the high hydraulic resistance encountered in inflating the catheter balloon with such contrast agents through an extremely small inflation lumen. Also, if the resistance is high, then the balloon may not deflate and this could result in catastrophic complications or inability to extricate the dilating system.

Poiseuille's law provides insight into the physical limitations of current catheter designs:

F(Flow Rate)=P₁−P₂/R, where:

R(Resistance to Flow)=8 μΩ/πr4.

In the above formulas, P₁ is the pressure at the inflation lumen entry point, P₂ is the pressure at the inflation lumen exit point, μ is the viscosity of the fluid within the inflation lumen, Ω is the length of the inflation lumen, and r is the radius of the inflation lumen. Based on these formulae, factors defining the minimal required diameter for the inflation lumen in a catheter include the catheter length and viscosity of the material being injected.

Conventionally, the only way one can determine if the balloon is fully inflated is by the injection of contrast agent into the balloon. A contrast agent has a defined viscosity. Based on Poiseuille's law, the only alternative to reducing viscosity of the fluid (thus allowing the radius of the inflation lumen to be decreased), is to decrease the length of the catheter. Since most transcatheter procedures are done via femoral or brachial arteries, an approach involving shortening the catheter length below 135 cm would result in the inability to reach the target lesion in many patients. It would thus be beneficial to have a system that that will allow balloon inflation with a gas or less viscous fluid, so the current physical limitations for miniaturizing catheter size could be overcome. Additionally, because determining balloon position is important to the success of most interventional procedures, there is a need for a catheter having a radiopaque balloon that avoids the drawbacks of previously-known designs.

In recognition of the foregoing drawbacks, U.S. Pat. No. 6,786,889 to Musbach, et al., describes a balloon having enhanced radiopacity achieved by texturing the surface of the balloon or providing a radiopaque ink on an exterior or interior surface of the balloon. One disadvantage to the design described in that patent is that the variations in texture may become less observable as the balloon size decreases. Moreover, depending upon the ink employed, the balloon may present biocompatibility issues.

In addition, attempts to reduce the profile of a catheter by reducing the wall thickness can lead to loss of “pushability” of the catheter, i.e., where the catheter has insufficient stiffness to be advanced over a guide wire when pushed from the proximal end. When coupled with the higher pressures required to inflate the balloon when using small lumens, reducing wall thickness also poses the risk of rupture during inflation.

In view of the foregoing, it would be desirable to provide a radiopaque interventional device having flexible walls with improved mechanical properties.

### SUMMARY

In view of the foregoing, the present invention provides a radiopaque interventional device having a flexible wall with improved mechanical properties.

In accordance with one aspect of this invention, an interventional device includes a balloon, at least a portion of which is formed from a composition including metal-coated carbon nanotubes dispersed in a polymer.

In some embodiments, the catheter includes an elongated shaft having proximal and distal ends and a lumen therebetween; and a balloon affixed to the elongated shaft near the distal end. In some embodiments, the balloon has a flexible wall that includes the composition. In other embodiments, the shaft includes the composition.

In preferred embodiments, each nanotube of a plurality of metal-coated nanotubes includes a carbon nanotube having an outer surface and a layer of metal disposed on at least a portion of the outer surface of the carbon nanotube. The layer of metal may be between about 1 nm and about 1 μm thick. Suitable metals include at least one of gold, silver, platinum, palladium, chromium, molybdenum, tungsten, manganese, technetium, rhenium, iron, ruthenium, osmium, cobalt, rhodium, iridium, nickel, copper, zinc, tin, and aluminum.

Preferably, the metal-coated nanotubes form a reinforcing web within the polymer, and may be substantially evenly dispersed in the polymer. The metal-coated nanotubes may be present in a concentration of less than 25 wt. % in the polymer, or less than 1 wt. % in the polymer, or less than 0.5 wt. % in the polymer, or less than 0.2 wt. % in the polymer.

In accordance with another aspect of the invention, a method of making a catheter including metal-coated nanotubes is provided. The method may include steps of: heating a polymer above a melting point of the polymer; dispersing a plurality of metal-coated nanotubes within the polymer to form a composition; and forming the composition into a catheter component. Alternatively, the method may include steps of: contacting a plurality of polymer particles with a plurality of metal-coated nanotubes; extruding the polymer particles and the metal-coated nanotubes to form a composition including the polymer and the metal-coated nanotubes; and forming the composition into a catheter component.

In some embodiments, forming into the catheter component the polymer having the plurality of metal-coated nanotubes dispersed therein includes extruding the polymer having the plurality of metal-coated nanotubes dispersed therein.

As further alternatives, the methods of manufacture may include forming the metal-coated nanotubes by depositing a layer of metal on at least a portion of the outer surface of each nanotube. Deposition of such a metal layer may be accomplished using at least one of solution chemical deposition, electrochemical deposition, chemical deposition, and physical deposition. Methods for physical deposition include at least one of evaporation, sputtering, and molecular beam epitaxy. In addition or in the alternative, depositing the layer of metal may include depositing a particulate metal or a metal precursor on the outer surface of each nanotube.

In accordance with another aspect of the present invention, a method of imaging interventional devices including metal-coated nanotubes is provided. The method includes: inserting into a body lumen a portion of a catheter, the catheter including a polymer with a plurality of metal-coated nanotubes dispersed therein; exposing the portion of the catheter to x-ray radiation; and obtaining an x-ray image of the portion of the catheter. In some embodiments, the method further includes positioning the catheter based on the x-ray image.

### BRIEF DESCRIPTION OF THE DRAWINGS

The advantages of the present invention will be apparent upon consideration of the following detailed description, taken in conjunction with the accompanying drawings, in which like reference characters refer to like parts throughout, and in which:

FIG. 1A illustrates a high-level plan view of an illustrative dilatation catheter of the present invention that includes a balloon formed of a composition including metal-coated nanotubes dispersed in a polymer.

FIG. 1B illustrates a longitudinal sectional view of the balloon of FIG. 1A.

FIG. 1C illustrates a plan view of an alternative embodiment of the balloon of FIG. 1A.

FIG. 1D illustrates a longitudinal sectional view of an alternative embodiment of the nanotube-reinforced balloon of FIG. 1A.

FIG. 2 is a flow chart describing steps in an illustrative method of positioning a dilatation catheter of the present invention using x-ray imaging.

FIG. 3A is a flow chart describing illustrative steps in a method of forming the balloon of FIGS. 1A-1B and 1D, according to some embodiments of the present invention.

FIG. 3B is a flow chart describing illustrative steps in an alternative method of forming the balloon of FIGS. 1A-1B and 1D, according to some embodiments of the present invention.

### DETAILED DESCRIPTION OF THE PREFERRED EMBODIMENT

The present invention provides interventional devices formed using compositions including metal-coated nanotubes dispersed in polymers, and methods of making and using same.

Specifically, embodiments of the invention provide components of interventional devices, such as balloons having a flexible wall made of a composition including a polymer with a plurality of metal-coated carbon nanotubes dispersed therein. Preferably, as least some of the nanotubes are at least partially coated with a radiopaque metal that imparts radio-opacity to device components formed using of the composition, thus allowing those device components to be imaged in vivo using fluoroscopy (x-ray imaging) without the need for a fluidic contrast agent. A physician may use the x-ray images to position the interventional device at a desired position within the subject's body. The physician then may expand the interventional device to restore patency to an occluded body lumen, e.g., an artery that is at least partially closed by a stenotic lesion.

In accordance with the principles of the present invention, the metal-coated nanotubes are present at a concentration sufficient to impart radiopacity to the component made from the inventive composition, The overall radiopacity of the component formed using the composition is a function of among other things, the radiopacity of the metal with which the nanotubes are coated, the extent to which the surfaces of the nanotubes are coated with the metal, the thickness of the metal on the nanotubes, and the thickness of the component. The metal-coated nanotubes may be present, for example, at a concentration of less than about 25% by weight of the composition, or less than 10 wt. %, or less than 5 wt. %, or less than 2 wt. %, or less than 1 wt. %, or less than 0.5 wt. %, or less than 0.2 wt. % of the composition. The metal-coated nanotubes may be substantially evenly dispersed within the polymer.

In some embodiments, the metal-coated nanotubes form a strong, interconnected, reinforcing web within the composition, thus enhancing the mechanical properties of the catheter component formed from that composition. Among other things, carbon nanotubes have a high tensile strength and elastic modulus, which increases the mechanical strength of the composition. This allows a radiopaque balloon wall to be fabricated having reduced dimensions and/or capable of withstanding higher pressures than would be possible in an otherwise identical balloon that lacks metal-coated nanotubes. For further details on dilatation catheters formed using nanotube-polymer compositions, see U.S. Provisional Patent Application No. 61/098,624, filed Sep. 19, 2008 and entitled “Interventional Devices Including Dilute Nanotube-Polymer Compositions, and Methods of Making and Using Same,” the entire contents of which are incorporated by reference herein.

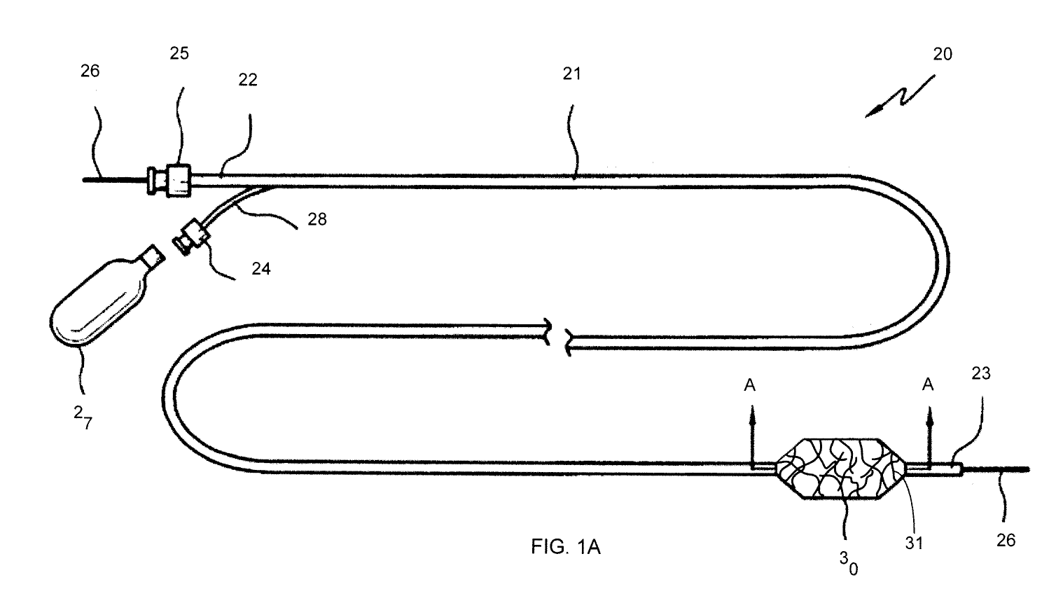

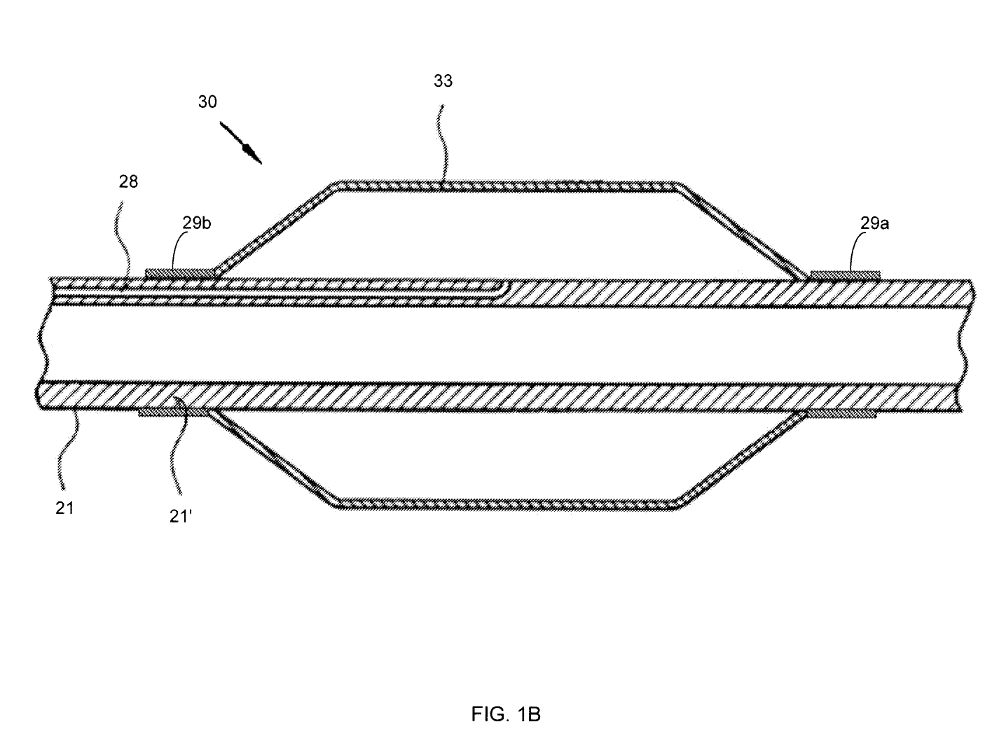

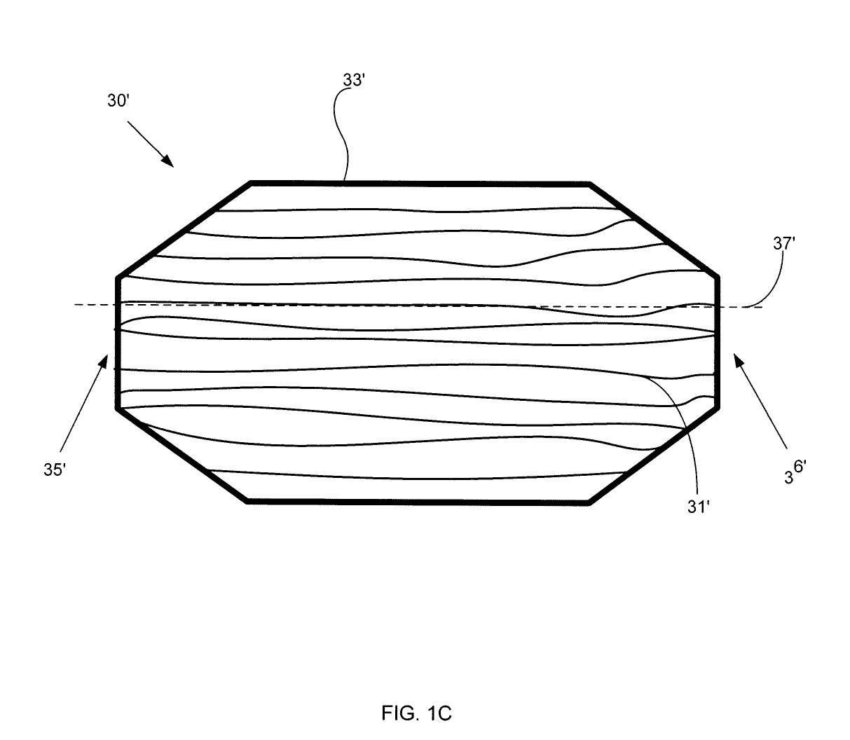

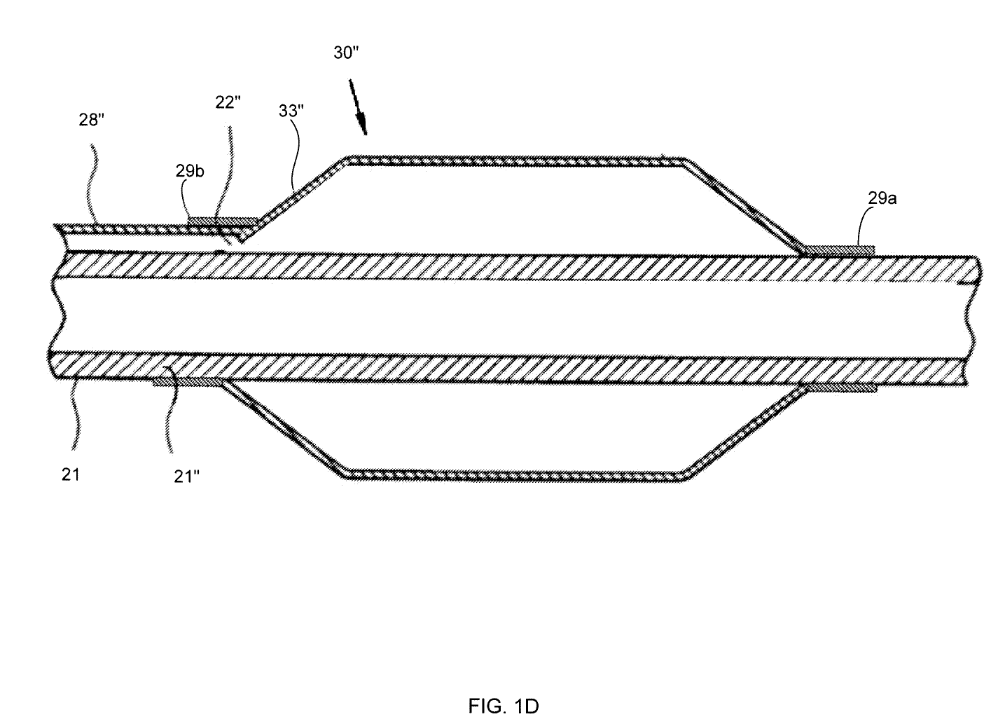

FIG. 1A illustrates a high-level plan view of a dilatation catheter constructed in accordance with some embodiments of the present invention. Dilatation catheter 20 includes shaft 21, proximal end 22, distal end 23, inflation port 24, manifold 25, guide wire 26, inflator 27, inflation lumen 28, and balloon 30 formed of a composition having a plurality of metal-coated nanotubes dispersed in a polymer. Inflator 27 is in fluidic communication with balloon 30 through inflation lumen 28 and may include a syringe or pump as is conventional for use with interventional devices. Inflator 27 supplies a pressurized fluid, e.g., a gas or liquid, to balloon 30.

As illustrated in greater detail in FIG. 1B, nanotube-reinforced balloon 30 includes flexible wall 33 affixed to shaft 21 via distal and proximal affixation zones 29a and 29b respectively. Inflation lumen 28 passes through wall 21′ of shaft 21, so that a distal end of inflation lumen 28 communicates with the space defined between the outer surface of shaft 21 and the inner surface of the flexible wall 33. The proximal end of inflation lumen 28 is coupled to inflation port 24. Inflation port 24 is coupled to inflator 27 (not shown in FIG. 1B).

Flexible wall 33 of balloon 30 includes a composition that includes a plurality of metal-coated carbon nanotubes 31 that are dispersed in a polymer. The metal-coated nanotubes may be present, for example, at a concentration of less than about 25% by weight of the composition, or less than 10 wt. %, or less than 5 wt. %, or less than 2 wt. %, or less than 1 wt. %, or less than 0.5 wt. %, or less than 0.2 wt. % of the composition. In accordance with various embodiments of the present invention, metal-coated nanotubes 31 may be present in a concentration of or less than 0.2 wt. %, or between 0.5 wt. % to 0.001 wt. % , or between 0.5 wt. % to 0.01 wt. %, or between 0.25 wt. % to 0.001 wt. %, or between 0.20 wt % to 0.001 wt. %, or between about 0.20 wt. % to 0.01 wt. %, or between 0.20 wt. % to 0.05 wt. %, or between 0.20 wt. % to 0.1 wt. %, or between 0.15 wt. % to 0.05 wt. %, or between 0.2 wt. % to 0.15 wt. %, or between 0.15 wt % to 0.10 wt. %, or between 0.10 wt. % to 0.05 wt. %, or about 0.2 wt %, or about 0.15 wt. %, or about 0.1 wt. %, or about 0.05 wt %, or about 0.025 wt %, or about 0.01 wt %, in the composition. For example, metal-coated nanotubes 31 may be present in a concentration of between 0.2 wt % and 0.01 wt. % in the composition. Or, for example, metal-coated nanotubes 31 may be present in a concentration of between 0.2 wt. % and 0.05 wt. % in the composition. Or, for example, metal-coated nanotubes 31 may be present in a concentration of between 0.15 wt. % and 0.05 wt. % in the composition. Other concentrations, and ranges of concentrations, are also contemplated.

In some embodiments, the composition from which wall 33 is formed consists essentially of metal-coated nanotubes 31 and polymer, so the weight percent of metal-coated nanotubes relative to the composition is the same as the weight percent of metal-coated nanotubes relative to the polymer. Those of skill in the art will recognize that small amounts of impurities may be present in the metal-coated nanotubes and/or polymer prior to mixing, such as residual catalyst, amorphous carbon, polymer initiators, and the like. Even if impurities are present in the metal-coated nanotubes and/or polymer, a composition in which only such nanotubes and such polymer are mixed together is still considered to consist essentially of metal-coated nanotubes and polymer. In alternative embodiments, the composition may include non-metal coated nanotubes (e.g., pristine nanotubes) in addition to the metal-coated nanotubes.

In other embodiments, other compounds may be present at low levels in the composition, such as a lubricant. As is known in the art, lubricant may be added to nanotube-polymer compositions in order to improve dispersion of the nanotubes within the polymer. In other embodiments, the composition excludes (i.e., contains no) lubricant. For example, depending on the particular application, the presence of lubricant may pose a risk of non-biocompatibility and/or a risk of structurally weakening wall 33. Other compounds optionally may be added to the composition, including pigments, plasticizers, dispersants, surfactants, contrast agents, and/or stabilizers. In some embodiments, each optional additive is present at a concentration of 1 wt. % or less in the composition.

In some embodiments, metal-coated nanotubes 31 are randomly oriented throughout flexible wall 33 of balloon 30. That is, each individual nanotube 31 may have an unconstrained orientation both relative to balloon 30 as a whole, through the thickness of wall 33, and relative to other nanotubes 31. Metal-coated nanotubes 31 may form an at least partially interconnected web throughout flexible wall 33, so that at least some coated nanotubes 31 contact at least one other coated nanotubes 31. Such contact may include a “crossing” contact in which the coated nanotubes cross at an angle of greater than zero degrees and less than or equal to 90 degrees. Alternatively, or additionally, such contact may include a “parallel” contact in which two or more coated nanotubes contact each other along at least a portion of their length (e.g., run in parallel contact for 5 or more nanometers). Those of skill in the art will recognize that such parallel contact between bare nanotubes naturally arises due to attractive van der Waals forces, and may be referred to for the extent of that contact as “nano-ropes.” Metal-coated nanotubes may experience analogous attractive forces and may at least partially aggregate into “ropes” of metal-coated nanotubes. While in many embodiments a plurality of metal-coated nanotubes 31, or even a majority of metal-coated nanotubes 31 (e.g., more than half of nanotubes 31) are not aggregated in the form of nanoropes but are instead individually dispersed throughout wall 33, in other embodiments a majority of coated nanotubes 31 may be present predominantly in the form of nanoropes. In some embodiments, aggregates of nanotubes are coated with metal.

The interconnectedness of nanotubes within wall 33 affects the mechanical properties of balloon 30, so it may be useful both to control and characterize such interconnectedness. For example, in some embodiments, a majority of nanotubes 31 contact at least one other nanotube 31, while in other embodiments, a majority of nanotubes 31 do not contact any other nanotubes 31, and in still other embodiments, substantially no nanotubes 31 contact other nanotubes 31, that is, each nanotube 31 is substantially isolated from each other nanotube by the polymer in wall 33.

While FIGS. 1A-1B illustrate metal-coated nanotubes 31 as having an unconstrained alignment relative to balloon 30, metal-coated nanotubes 31 alternatively may be aligned relative to balloon 30, which may inhibit one or more potential modes of tearing of balloon 30. For example, FIG. 1C illustrates an embodiment in which metal-coated nanotubes 31′ are aligned longitudinally relative to balloon 30′. The longitudinal orientation of metal-coated nanotubes 31′ may inhibit potential radial modes of tearing of balloon 30′. In another embodiment (not illustrated), metal-coated nanotubes 31′ are aligned radially relative to balloon 30′, which may inhibit potential longitudinal modes of tearing of balloon 30′.

In the embodiment illustrated in FIG. 1C, at least a subset of metal-coated nanotubes 31′ generally extend between first end 35′ of balloon 30′ and second end 36′ of balloon 30′. However, not all of metal-coated nanotubes 31′ need extend the entire distance between first and second ends 35′, 36′; for example, some or all of metal-coated nanotubes 31′ may be longitudinally oriented relative to balloon 30′ but may only extend a portion of the distance between first and second ends 35′, 36′. Even if no metal-coated nanotubes 31′ extend the entire distance between ends 35′ and 36′, the wall 33′ is still reinforced. Metal-coated nanotubes 31′ also need not be precisely oriented relative to balloon 30′. For example, the orientation of each individual metal-coated nanotube 31′ may deviate by more than 1%, more than 2%, more than 5%, or even more than 30% from imaginary line 37′, which represents an orientation that is longitudinal relative to balloon 30′. Radially aligned metal-coated nanotubes may have similar length and/or alignment as described for longitudinally aligned metal-coated nanotubes.

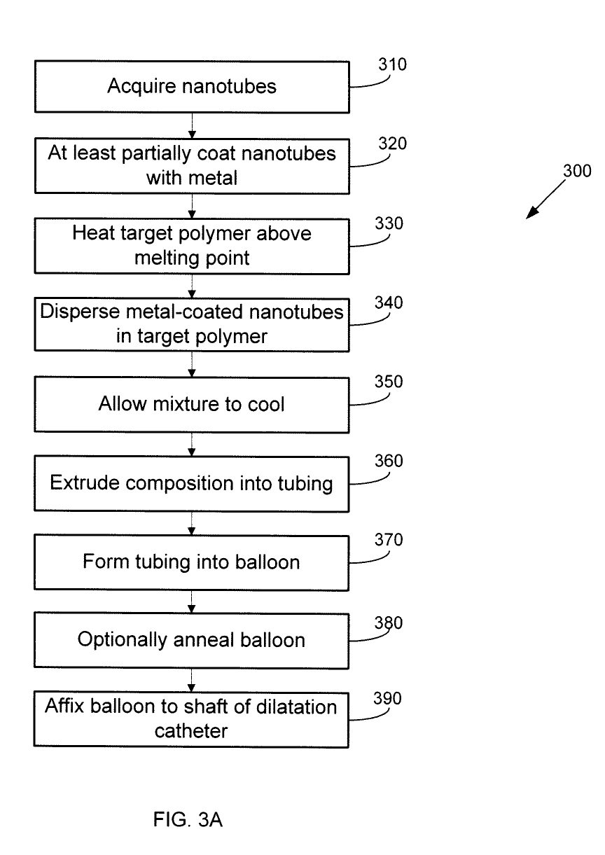

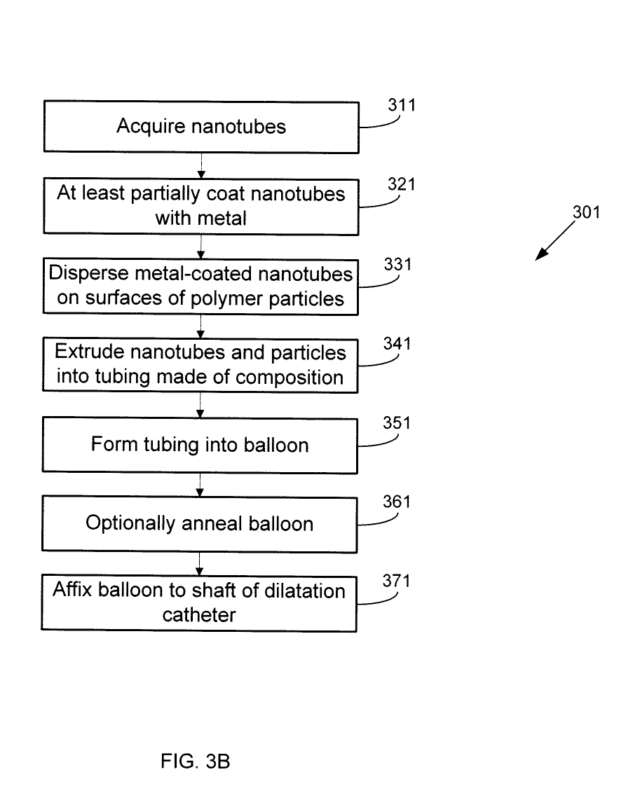

Metal-coated nanotubes 31′ may be aligned longitudinally and/or radially relative to balloon 30′ using any suitable technique, e.g., gel spinning or electrospinning. In gel spinning, metal-coated nanotubes 31′ are dispersed within a suitable melted polymer (e.g., as described below with respect to FIG. 3A). The melted nanotube/polymer mixture is extruded through a suitably shaped die and the extruded end drawn, thereby causing chains of the polymer and the metal-coated nanotubes to align substantially parallel to the direction of extrusion. To form balloon 30′ having longitudinally oriented metal-coated nanotubes 31′, the die preferably is cylindrically shaped and includes a mandrel for forming a lumen within balloon 30′. To instead form balloon 30′ having radially oriented metal-coated nanotubes 31′, the die is shaped to extrude a sheet or ribbon; after extrusion, the sheet or ribbon may be looped into a cylinder and the edges sealed (e.g., with heat and pressure) to form balloon 30′. Electrospinning includes applying an electrical field during extrusion that orients the metal-coated nanotubes.

In an alternative embodiment of balloon 30, designated 30″ in FIG. 1D, inflation lumen is disposed against wall 21″ of shaft 21. Port 22″ exists in flexible wall 33″, and inflation lumen 28″ may pass through port 22″ and terminate within balloon 30″. Alternatively, the distal end of inflation lumen 28″ may be affixed to flexible wall 33″ such that it provides communication through port 22″.

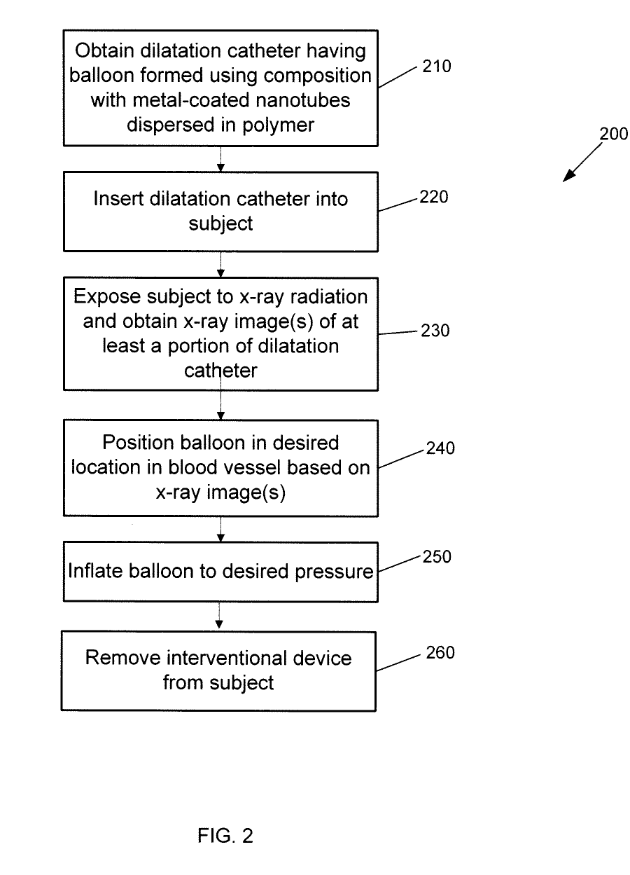

FIG. 2 illustrates steps in method 200 of using x-ray imaging to position and deploy a dilatation catheter including a balloon formed using a composition that includes metal-coated nanotubes dispersed in a polymer, according to some embodiments of the present invention. First, a dilatation catheter constructed in accordance with an embodiment of the present invention, which includes a balloon formed of a composition including metal-coated nanotubes dispersed in a polymer, is obtained (210).

The dilatation catheter is inserted into the subject (220). For example, the dilatation catheter may be inserted into a blood vessel (e.g., vein or artery) of the subject.

The subject then is exposed to x-ray radiation, and one or more x-ray images are obtained of at least a portion of the dilatation catheter (e.g., the balloon). In some embodiments, a series of still x-ray images are obtained, while in other embodiments, a movie is obtained in real-time.

The physician then positions the balloon at a desired location in the blood vessel (e.g., adjacent a stenotic lesion) based on the obtained x-ray image(s) (240). Specifically, the metal-coated nanotubes within the dilatation catheter act as a contrast agent that imparts radiopacity to the balloon, allowing it to be viewed (e.g., in real-time) using x-ray imaging.

The physician then inflates the balloon of the dilatation catheter with fluid to a desired pressure (250). The pressure may be sufficient to disrupt a stenotic lesion, while at the same time inflating the balloon to a diameter that is sufficient to expand, but not over-expand, the blood vessel. In some embodiments, the pressure to which the balloon is inflated is in the range of about 4 to 16 atm, or about 4 to 8 atm, or about 4 to 6 atm, or about 6 to 8 atm, or about 8 to 16 atm, or about 8 to 12 atm, or about 10 to 12 atm, or about 12 to 16 atm, or about 16 to 18 atm, or about 10 to 35 atm, or 10 to 20 atm, or 15 to 25 atm, or 20 to 30 atm, or 30 to 35 atm. The dilatation catheter is then removed from the subject (260).

FIGS. 3A-3B illustrate exemplary methods of forming the balloon 30 of FIGS. 1A-1B and 1D, according to some embodiments.

Referring first to method 300 of FIG. 3A, nanotubes are acquired (310), for example from commercially available sources, or are fabricated. The nanotubes may include single-walled carbon nanotubes (SWNT) and/or multi-walled carbon nanotubes (MWNT). The carbon nanotubes may include conducting and/or semiconducting nanotubes. The carbon nanotubes may be “pristine,” that is, not functionalized, derivitized, or otherwise modified (e.g., including substantially no atoms other than carbon). Alternatively, one or more of the carbon nanotubes may be covalently or non-covalently functionalized or derivitized to have desired therapeutic properties, to enhance their dispersion in the polymer, or to enhance the deposition of metal thereon. Generally, derivatization of a carbon nanotube will refer to the covalent bonding of one or more functional groups to the ends and/or sidewall of the carbon nanotube. The nanotubes may also, or alternatively, be filled with a cargo such as a metal, e.g., using techniques and materials known in the art.

Other types of materials may be used in the balloon, either in addition to the nanotubes, or instead of the nanotubes. For example, inorganic nanotubes (e.g., tungsten disulfide, boron nitride, silicon, titanium dioxide, molybdenum disulfide, copper, or bismuth nanotubes) may be used. Or, for example, graphene fibers may be used to reinforce the flexible wall of the balloon. Or, for example, fibers of KEVLAR® (poly paraphenylene terephthalamide), TEFLON® (polytetrafluoroethylene), TERLON® (poly(p-phenylenebenzobisthiazole)), ZYLON® (poly(p-phenylene-2,6-benzobisozazoles), polyether block amides, and VECTRAN® (liquid crystal polymer) may be used in the composition. The selected material(s) are capable of being at least partially coated with a metal, which renders them metal-coated and thus radiopaque.

In accordance with the present invention, at lease some of the nanotubes (and/or other materials) are at least partially coated with metal (320). In some embodiments, the metal includes one or more of the following elements: scandium, titanium, zirconium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, aluminum, gallium, indium, germanium, tin, antimony, lead, or bismuth. In some embodiments, the metal includes gold, silver, platinum, palladium, and/or aluminum. The metal may also include other elements.

The metal may be deposited on the carbon nanotube ends, interior and/or exterior surfaces by solution chemical deposition, electrochemical deposition, chemical deposition, physical deposition by evaporation, sputtering, molecular beam epitaxy, electron beam deposition, electrochemical deposition, such as electroplating and electrodeposition, and combinations thereof.

In some embodiments, atoms or molecules, such as polymers, may non-covalently attach to single-wall carbon nanotubes and adhere through wrapping, such as polymer wrapping, or electrostatic attraction, such as by polarization forces or by van der Waals forces. Metal and metal-containing species may attach to the non-covalently derivatized nanotube due to the chemical attraction provided by the non-covalent derivatizing agent. Subsequently, the non-covalent derivatizing agent may be removed (for instance by pyrolysis) to leave the metal in contact with the nanotube. Both covalent derivatization and non-covalent attachment of other chemical species may facilitate the deposition of metal onto the carbon nanotube.

In one embodiment, the metal may be deposited on the carbon nanotubes in a small (i.e. Angstrom- or nanometer-scale) particulate form, and optionally annealed to form an at least partially contiguous coating on the nanotubes.

In other embodiments, the metal may be deposited on the carbon nanotubes using a metal precursor. For example, the carbon nanotubes may derivatized with a functional group, and a metal-containing precursor compound reacted or complexed with the functional group. For example, the functional groups on derivatized nanotubes react or complex with the metal or metal precursor. Metal precursor deposition techniques include, but are not limited to, solution deposition, incipient wetness, ion exchange, and combinations thereof. After deposition, the metal precursor may be treated by chemical or physical processes to convert the metal into a metallic state. Such treatments include, but are not limited to, gaseous reduction, chemical reactive reduction, oxidation, heat treatment, chemical reaction with other compounds, and combinations thereof.

The metal may be deposited to an extent that is sufficient to impart radiopacity to the nanotubes for the particular application of the metal-coated nanotubes (e.g., to impart radiopacity to the particular component to be formed from the composition including those coated nanotubes). For example, the metal may be deposited so that it coats a portion of, or substantially the entirety of, the outer surface of each of the nanotubes. Or, for example, the metal may be deposited so that it fills at least a subset of interstices between nanotubes, e.g., interstices between nanotubes in a nanorope.

A suitable polymer then is heated above its melting point (330). Suitable polymers include elastomers such as EPDM, epichlorohydrin, nitrile butadiene elastomers, and silicones, epoxies, fluoropolymers such as polytetrafluoroethylene (PTFE, trade name TEFLON®), isocyanates, nylon, poly(acrylic acid), polyamides such as PEBAX® (tradename for polyether block amide, available from Arkema, Colombes, France), polybutene, polybutylene naphthalate, polycaprolactone, polycarbonate, poly(dimethylsiloxane), polyester, polyether, polyethylene, polyethylene naphthalate, polyethylene terephthalate (PET, trade name DACRON®, DuPont, Wilmington, Del.), polyimides, polyisobutene, polyisoprene, poly(methacrylic acid), polyolefin, polyoxide, polypropylene, polysiloxane, polystyrene, polysulfide, polyurea, polyurethane, poly(vinyl acetate), poly(vinyl alcohol), poly(vinyl chloride) (PVC), poly(vinyl pyridine), poly(vinyl pyrrolidone), urethanes, and copolymers thereof and combinations thereof, or other polymer that is biocompatible, in which nanotubes disperse, and that when mixed with nanotubes is capable of being formed into a balloon having desired mechanical properties. Examples of some suitable polymers may be found in U.S. Pat. No. 5,871,468, entitled “Medical Catheter With a High Pressure/Low Compliant Balloon,” the entire contents of which are incorporated herein by reference. Copolymers of tetrafluoroethylene with ethylene, cholorotrifluoroethylene, perfluoroalkoxytetrafluoroethylene, or fluorinated propylenes such as hexafluoropropylene also may be used. In some embodiments, the polymer is polar, or is at least partially polar, which in some embodiments may aid in dispersing the nanotubes throughout the polymer. For further details, see U.S. Pat. No. 6,936,653, the entire contents of which are incorporated herein by reference. In one example, the polymer includes, or consists essentially of, nylon, e.g., PA-12 (such as GRILAMID-L25®, available from EMS-Chemie AG, Reichenauerstrasse Switzerland, or L1800®, available from Evonik Industries, Essen Germany). In another example, the polymer includes, or consists essentially of, PEBAX®. In another example, the polymer includes, or consists essentially of, polyurethane.

Still referring to FIG. 3A, the metal-coated nanotubes then are mixed into the melted polymer while the polymer is maintained at a temperature above its melting point (340). Specifically, the metal-coated nanotubes are added to the polymer in an amount sufficient for the balloon eventually formed of the resulting composition to have sufficient mechanical properties to withstand pressurization. For example, the metal-coated nanotubes may be added to the polymer in an amount of less than about 5 wt. %, or less than about 4 wt. %, or less than about 3 wt. %, or less than about 2 wt. %, or less than about 1 wt. %, or less than about 0.5 wt. %, or less than about 0.2 wt. %, or less than about 0.1 wt. %. In one example, the metal-coated nanotubes are added to the polymer in an amount between about 0.05 wt. % and about 0.5 wt. %.

In some embodiments, no other materials are added to the composition other than the nanotubes and the polymer. In other embodiments, at least one other material (such as a lubricant, solvent, or surfactant) that aids dispersion of the nanotubes in the polymer is added. Typically, it is useful to reduce or avoid the presence of particulates in the composition because such particulates may reduce the mechanical strength and reliability of the balloon fabricated from the composition. In some embodiments, the nanotubes and/or polymer are selected to be of sufficient purity that substantially no particulates are present that would otherwise reduce the mechanical strength and reliability of a balloon formed from the composition.

The metal-coated nanotubes, either alone or with pristine nanotubes, may be thoroughly and substantially evenly distributed (dispersed) throughout the polymer using mechanical agitation, for example, using a material compounder, moving a container holding the nanotube-polymer solution, stirring the nanotube-polymer solution, or by maintaining the nanotube-polymer solution above the polymer melting temperature for an extended period of time. The mechanical agitation alternately may be performed using sonic energy, e.g., using an ultrasonic homogenizer (sonicator), or using low-frequency high-energy sonic energy, such as a ResonantAcoustic® mixer available from Resodyn Inc., Butte, Mont.

Those of skill in the art will recognize that nanotubes tend to “clump” together due to van der Waals forces and other attractive forces, and thus may form “nanoropes” made up of bundles of nanotubes. The polymer/metal-coated nanotube solution may therefore include one or more of such nanoropes. While in many embodiments the solution includes a relatively even distribution of nanotubes, the presence of an occasional nanorope is not believed to detrimentally affect the properties of the nanotube-reinforced balloon eventually formed from the solution. Some embodiments include a relatively even distribution of nanoropes in the polymer.

Once the nanotubes are distributed in the polymer to a satisfactory degree, the polymer/metal-coated nanotube solution is allowed to cool (350), forming a polymer/metal-coated nanotube composition. The polymer may be cross-linked at this point, or at another suitable point, using electromagnetic (e.g., e-beam) radiation.

The composition is then extruded into tubing (360), which is then formed into a balloon (370), for example, as described below with respect to FIG. 4B. It should be understood that prior to forming the balloon, the nanotube-polymer composition is optionally pelletized and re-extruded or otherwise formed using known techniques.

The balloon optionally is annealed (380), which may increase the burst pressure of the balloon. In one example, the balloon is annealed by raising the temperature of the balloon to a controlled level below the melt temperature of the composition, but high enough to give the polymer molecules the ability to move slightly. For a composition containing PA-12, the balloon may be held at a temperature of 120-180° F. for 30-120 minutes. Without wishing to be bound by a theory, it is believed that the annealing process relieves some of the residual stresses from balloon fabrication (e.g., from blow-molding) and allows some residual polymeric crystallization to take place, which may be a strengthening mechanism. Annealing may provide the balloon with an additional 5-15% increase in burst pressure and a reduction in the variation of the mechanical properties of the balloon (e.g., a reduced standard deviation in the burst pressure and the compliance of the balloon).

The nanotube-reinforced balloon is affixed to the shaft of an interventional device such as a dilatation catheter (390), for example dilatation catheter 20 illustrated in FIG. 1A, using conventional affixation methods.

Optionally, other catheter components may be formed of the polymer/metal-coated nanotube composition. For example, the composition may be used to form a catheter shaft, such as catheter shaft 21 of dilatation catheter 20 illustrated in FIG. 1A. Metal-coated nanotubes, if used in shaft 21, may provide increased axial strength and pushability to dilatation catheter 20, thereby allowing shaft 21 to be produced with reduced dimensions, while at the same time reducing the likelihood of kinking, binding, or similar problems that may conventionally accompany the reduction in dimensions of dilatation catheter parts. Or, for example, the metal-coated nanotubes may provide “steerability” to shaft 21 and/or guide wire 26. For further details, see U.S. Patent Publication No. US 2007/0100279 A1, entitled “Radiopaque-Balloon Dilatation Catheter and Methods of Manufacture,” the entire contents of which are incorporated by reference herein. The type of nanotube and the type of polymer used in balloon 30, and in other parts of dilatation catheter 20, may be selected independently of one another. The composition also may be used to form other types of components, e.g., components in interventional devices such as catheters that do not include a balloon, or guidewires.

In embodiments in which both balloon 30 and shaft 21 are made using a polymer/metal-coated nanotube composition, the metal-coated nanotubes may be used to enhance bonding between balloon 30 and shaft 21. Specifically, nanotubes have a propensity to lock together when brought near each other. Balloon 20 and shaft 21 may be bonded together using a technique that allows the nanotubes in the two components to lock together, for example, by bringing the two components adjacent each other and then heating the polymer/metal-coated nanotube composition just higher than the melting point of the polymer. While the polymer is heated, nanotubes in one component may move through the composition and lock together with nanotubes in the other component. Electromagnetic fields optionally may be used to selectively orient the metal-coated nanotubes and/or enhance the transport of metal-coated nanotubes from one component to the other.

FIG. 3B illustrates alternative method 301 of forming a balloon using a dilute nanotube-polymer composition, such as balloon 30 illustrated in FIGS. 1A-1B and 1C.

Nanotubes are acquired (311) and at least a portion of the nanotubes are at least partially coated with metal (321), e.g., as described above with reference to steps 310 and 320 of FIG. 3A, respectively.

The metal-coated nanotubes then are dispersed on the surfaces of solid polymer particles (331). For example, the polymer may be in the form of pellets, granules, grains, beads, microcapsules, microspheres, nanospheres, microparticles, micropellets, nanoparticles, or a powder (collectively referred to herein as “particles”). The particles may have sizes ranging between, for example, 1 nm and 250 μm, e.g., 10 nm to 100 μm, or 100 nm to 10 μm, or 10 μm to 250 μm. Sizes larger than 250 μm also may be used, for example, between 250 μm and 5 mm, e.g., 1 mm to 5 mm. In one example, a polymeric powder having particle sizes between 1 μm and 100 μm is used, e.g., having particle sizes between 5 and 60 μm. Without wishing to be bound by a theory, it is believed that small polymeric particle sizes may enhance the dispersion of metal-coated nanotubes in the composition by increasing the available surface area to which the coated nanotubes adhere, and thus reducing the likelihood that the coated nanotubes agglomerate together into nanoropes.

The polymer may be one of the polymers listed above with reference to FIG. 3A. In one example, the polymer includes, or consists essentially of, nylon, e.g., PA-12. In another example, the polymer includes, or consists essentially of, PEBAX®. In another example, the polymer includes, or consists essentially of, polyurethane. The polymer may be cross-linked using electromagnetic (e.g., e-beam) irradiation to modify its properties.

The metal-coated nanotubes may be dispersed on the surfaces of the polymer particles using several suitable techniques, some examples of which are provided below.

In some embodiments, the metal-coated nanotubes are added in a solid state (e.g., as a powder) directly to the particulate polymer, and the mixture then mechanically agitated to coat the outer surfaces of the polymer particles with metal-coated nanotubes. Such mechanical agitation can include, for example, manually moving a container holding the nanotube-particulate polymer mixture, using a material compounder, or stirring the nanotube-particulate polymer mixture. The mechanical agitation alternately may be performed using sonic energy, e.g., using an ultrasonic homogenizer (sonicator), or using low-frequency high-energy sonic energy, such as a ResonantAcoustic® mixer available from Resodyn Inc., Butte, Mont.

In other embodiments, the metal-coated nanotubes are added in a solubilized state (e.g., dispersed in a suitable solvent and/or surfactant) to the particulate polymer. For example, the solubilized metal-coated nanotubes may be sprayed onto the surface of the polymer particles, and the liquid subsequently removed to leave the metal-coated nanotubes on the surface of the polymer particles. The polymer particles may be stationary while the metal-coated nanotubes are applied to them, or they may be agitated (e.g., tumbled) while the metal-coated nanotubes are applied to them. Or, for example, the polymer particles are added to a liquid containing the metal-coated nanotubes, the mixture agitated (e.g., as described above), and the liquid then removed to leave the nanotubes on the surface of the polymer particles. In such embodiments, the liquid can be removed in any suitable manner, for example, by heating the mixture to drive off the liquid, or allowing the liquid to evaporate at ambient temperature. The mixture subsequently may be “powderized” to separate the nanotube-coated particles from one another, thus inhibiting agglomeration of metal-coated nanotubes. Powderizing may be performed by mechanical agitation, e.g., by sonication or other agitation mechanism described above. Alternatively, the solution of nanotube-coated particles may be spread (e.g., by spraying) onto a surface to enhance evaporation of the liquid while simultaneously inhibiting agglomeration of metal-coated nanotubes.

In still other embodiments, the metal-coated nanotubes are added in a gaseous state to the particulate polymer. For example, the metal-coated nanotubes may be aerosolized, and the polymer particles exposed to the aerosol, e.g., by spraying the polymer particles into a container holding the aerosolized nanotubes. The metal-coated nanotubes are attracted to and adhere to the surfaces of the polymer particles, which subsequently may be collected, e.g., by allowing them to fall to the bottom of the container.

The polymer optionally may be softened (e.g., using heat or a suitable solvent) to enhance adhesion of the metal-coated nanotubes to the surface of the polymer particles. The composition of the polymer and/or the surface characteristics of the polymer particles also may be selected to enhance adhesion of the nanotubes to the surface of the polymer particles. The size of the particles may also be selected to (a) enhance the attraction of the metal-coated nanotubes to the particle surfaces, and (b) enhance the metal-coated interconnectedness of the nanotubes within the composition. The parameters (a) and (b) may not necessarily depend on the particle size in the same way, so the particle size may be selected based on a balance between (a) and (b) for the desired purpose of the composition.

In some embodiments, a multi-step process optionally may be used to first encapsulate metal-coated nanotubes with a thin layer of a first polymer, and the coated metal-coated nanotubes then mixed with a second polymer that may be the same or different from the first polymer. For example, a polymer may be aerosolized, and the metal-coated nanotubes exposed to the aerosol, e.g., by spraying the aerosolized polymer particles into a container holding the nanotubes. The polymer-coated metal-coated nanotubes then may be mixed with the same or a different polymer. For example, nylon-coated nanotubes may be dispersed in PEBAX®. For further details on certain methods of coating nanotubes with polymers, see the following patent references, the entire contents of each of which are incorporated herein by reference: U.S. Pat. No. 7,264,876 and U.S. Pat. No. 7,008,563.

After the metal-coated nanotubes are dispersed on the surfaces of the polymer particles, the particles are extruded into tubing formed of the inventive composition (341). The particles may be directly extruded into the tubing, or may be first pelletized and the pellets extruded into the tubing.

In an exemplary extrusion process, the mixture of particulate polymer and metal-coated nanotubes is loaded into a heated, barrel-shaped container to dry them. The mixture falls into an extruder and as the polymer particles are heated above their melting point they melt and liquefy, and a rotating screw in the extruder mixes the polymer and dispersed nanotubes into a substantially homogeneous blend. The liquefied mixture then is pumped through the extruder, which has a nozzle at one end, with an airtube centered in the nozzle. The airtube assists in controlling the finished dimensions of the tubing. The liquefied mixture exits the extruder as a long tube, the outside diameter of which is defined by the die diameter, and the inside diameter of which is defined by the airtube. The tube is pulled from the nozzle and through a cooling bath by a mechanical puller, thus solidifying the tubing. The tubing may be cut to individual lengths. In one example, the particles are extruded using a conventional extruder, such as a Microtruder (Randcastle Extrusion Systems, Inc., Cedar Grove, N.J.).

The tubing is formed into a balloon (351). In one embodiment, each balloon is formed from an individual length of extruded tubing using a two-step process. The first step is necking the tube into a balloon parison. During this process, the tubing is heated and stretched. The heating is controlled below the melting point, such that it lowers the yield strength for the localized material that is being stretched.

The second step of balloon formation is blow-molding. The balloon parison is inserted into a heated mold that is mounted in a balloon blow machine. There are various molds that correspond to different finished diameters and lengths of balloons; the particular mold is selected based on the desired characteristics of the finished balloon. Next, one end of the balloon parison is clamped shut, and the open end is connected to a supply of compressed nitrogen or other non-reactive gas. The compressed nitrogen then is actuated, which pressurizes the balloon parison to a constant internal pressure while the heated mold warms the parison. The balloon blow machine includes one or more sensors that determine when the parison has reached a suitable temperature for the next step of blow-molding. After the warm-up, the parison may be stretched slightly by the pressure of the compressed nitrogen. The nitrogen pressure then is increased to a higher pressure for a specified amount of time. During this time, the heated parison stretches to conform to the shape of the mold, thus forming the balloon. The balloon is cooled and removed from the mold.

The balloon thus formed has a flexible wall formed of a composition including metal-coated nanotubes dispersed in a polymer. As discussed above with reference to FIG. 3A, the balloon optionally may be annealed (361) to further enhance its mechanical characteristics. The balloon is affixed to the shaft of an interventional device such as a dilatation catheter (371).

Other methods may be used to form the composition and/or balloon. For example, the metal-coated nanotubes may be dispersed in a monomer (and optionally also a solvent), and the monomer polymerized to form the composition. The composition then may be formed into a balloon, e.g., as described above.

Examples of suitable blood vessels for treatment using the balloons and catheters described herein include coronary, renal, iliac, femoral, distal leg and carotid arteries as well as saphenous vein grafts, synthetic grafts and arteriovenous shunts used for hemodialysis. It is contemplated that the balloons and catheters described herein have applicability for use with other types of body passageways, including, but not limited to, urethra, prostate, prostatic urethra, esophagus, fallopian tubes, rectum, intestines, bronchi, kidney ducts, wind pipe, pancreatic ducts, gall bladder ducts, biliary ducts, brain parenchyma, and the like.

Although various embodiments of the present invention are described above, it will be evident to one skilled in the art that various changes and modifications may be made without departing from the invention. It is intended in the appended claims to cover all such changes and modifications that fall within the true spirit and scope of the invention.

What is claimed:

1. A catheter comprising a polymer with a plurality of metal-coated nanotubes dispersed therein.

2. The catheter of claim 1, wherein the catheter comprises:
an elongated shaft having proximal and distal ends and a lumen therebetween; and
a balloon affixed to the elongated shaft near the distal end.

3. The catheter of claim 2, wherein the balloon has a flexible wall, the flexible wall comprising the polymer with the plurality of metal-coated nanotubes dispersed therein.

4. The catheter of claim 2, wherein the shaft comprises the polymer with the plurality of metal-coated nanotubes dispersed therein.

5. The catheter of claim 1, wherein each nanotube of the plurality of metal-coated nanotubes comprises a carbon nanotube having an outer surface and a layer of metal disposed on at least a portion of the outer surface of the carbon nanotube.

6. The catheter of claim 5, wherein the layer of metal is between about 1 nm and about 1 μm thick.

7. The catheter of claim 5, wherein the metal includes at least one of gold, silver, platinum, palladium, chromium, molybdenum, tungsten, manganese, technetium, rhenium, iron, ruthenium, osmium, cobalt, rhodium, iridium, nickel, copper, zinc, tin, and aluminum.

8. The catheter of claim 1, wherein the metal-coated nanotubes form a reinforcing web within the polymer.

9. The catheter of claim 1, wherein the metal-coated nanotubes are substantially evenly dispersed in the polymer.

10. The catheter of claim 1, wherein the metal-coated nanotubes are present in a concentration of less than 25% w/w in the polymer.

11. A method of making a catheter, the method comprising:
heating a polymer above a melting point of the polymer;
dispersing a plurality of metal-coated nanotubes within the polymer; and
forming into a catheter component the polymer having the plurality of metal-coated nanotubes dispersed therein.

12. The method of claim 11, wherein the catheter component comprises at least one of a catheter shaft and a dilatation balloon.

13. The method of claim 12, wherein forming into the catheter component the polymer having the plurality of metal-coated nanotubes dispersed therein comprises extruding the polymer having the plurality of metal-coated nanotubes dispersed therein.

14. The method of claim 11, further comprising forming the metal-coated nanotubes by depositing a layer of metal on at least a portion of the outer surface of each nanotube.

15. The method of claim 14, wherein depositing the layer of metal comprises at least one of solution chemical deposition, electrochemical deposition, chemical deposition, and physical deposition.

16. The method of claim 15, wherein said physical deposition comprises at least one of evaporation, sputtering, and molecular beam epitaxy.

17. The method of claim 14, wherein depositing the layer of metal comprises depositing a particulate metal or a metal precursor on the outer surface of each nanotube.

18. An imaging method comprising:
inserting into a body lumen a portion of a catheter, the catheter comprising a polymer with a plurality of metal-coated nanotubes dispersed therein;
exposing the portion of the catheter to x-ray radiation; and
obtaining an x-ray image of the portion of the catheter.

19. The method of claim 18, further comprising positioning the catheter based on the x-ray image.

20. The method of claim 18, wherein the catheter comprises:
an elongated shaft having proximal and distal ends and a lumen therebetween; and
a balloon affixed to the elongated shaft near the distal end.

21. The method of claim 20, wherein the balloon has a flexible wall, the flexible wall comprising the polymer with the plurality of metal-coated nanotubes dispersed therein.

22. The method of claim 20, wherein the shaft comprises the polymer with the plurality of metal-coated nanotubes dispersed therein.

23. The method of claim 18, wherein the metal-coated nanotubes form a reinforcing web within the flexible wall.

24. The method of claim 18, wherein the metal-coated nanotubes are substantially evenly dispersed in the polymer.

25. The method of claim 18, wherein the metal-coated nanotubes are in a concentration of less than 25% w/w in the polymer.
